(19) United States
(12) Patent Application Publication
(10) Pub. No.: US 2026/0159768 A1
(43) Pub. Date: Jun. 11, 2026

(54) Steam Cracking a Hydrocarbon Feed Comprising Arsenic

(71) Applicant: ExxonMobil Chemical Patents Inc., Baytown, TX (US)

(72) Inventors: Rodney S. Smith, Edinburgh (GB); Michael A. Radzicki, Houston, TX (US); Donald J. Norris, London, Ontario (CA)

(21) Appl. No.: 18/704,356

(22) PCT Filed: Nov. 21, 2022
(86) PCT No.: PCT/US2022/080241

§ 371 Date: Apr. 24, 2024

Related U.S. Application Data
(60) Provisional application No. 63/287,625, filed on Dec. 9, 2021.

(51) Int. Cl. C10G 55/04; C07C 4/04; C07C 5/09; C10G 9/36; C10G 31/08; C10G 45/08; C10G 45/36; C10G 67/04; C10G 67/14

(52) U.S. Cl. CPC C10G 55/04; C07C 4/04; C07C 5/09; C10G 9/36; C10G 31/08; C10G 45/08; C10G 45/36; C10G 67/04; C10G 67/14; C07C 2523/882; C07C 2523/883; C10G 2300/202; C10G 2300/205; C10G 2400/06; C10G 2400/20

(57) ABSTRACT

A process for producing light olefins from a hydrocarbon feed comprising arsenic at an initial quantity. The process can comprise one of more of: introducing the hydrocarbon feed to a desalter to produce a desalted hydrocarbon feed having a reduced quantity of arsenic: preheating the desalted hydrocarbon feed; introducing the preheated hydrocarbon feed to a flash separation vessel to produce an overhead fraction and a bottoms fraction; introducing the overhead fraction and steam to a radiant section of the steam cracker operated under steam cracking conditions to produce a. steam cracked effluent; and separating the steam cracked effluent to obtain a steam cracker tar, a steam cracker gas oil, a naphtha, cut, and a process gas stream; and recovering an olefin product stream from the process gas stream. Arsenic quantities in various streams in the process can be calculated and controlled.

### CROSS-REFERENCE TO RELATED APPLICATION

This application claims priority to and the benefit of U.S. Provisional Application No. 63/287,625 having a filing date of Dec. 9, 2021, the disclosure of which is incorporated herein by reference in its entirety.

### FIELD

The present disclosure relates to processes for producing olefins from a hydrocarbon feed comprising arsenic. In particular, this process relates to steam cracking heavy hydrocarbon-containing and arsenic-containing feed, such as crude, for making light olefins such as ethylene and propylene.

### BACKGROUND

Historically, steam cracker liquid feedstocks used to produce olefins have come from refinery process streams such as naphtha, gas oil, resids and other cuts. The refining process removes many of the contaminants that were present in the raw crude feedstocks rendering thus produced steam cracker liquid feedstocks relatively low in arsenic particularly suitable for steam cracking.

Over time, growth rates in demand for olefins have exceeded the growth rates in demand for refinery fuels, and the growth trend for olefins is expected to continue into the future. In recent years, the size of steam crackers has greatly increased over the growth of fuel refineries. It has become increasingly desirable to utilize heavy hydrocarbon-containing feeds (e.g., crudes) as feed to steam crackers to produce olefins. The use of crude oil in steam cracking would remove dependency on the limited supply and relatively high costs of the various refinery fuel cuts. The economics for processing the raw feedstocks directly in a steam cracker can be advantaged over refinery process streams both in terms of improved operation and investment cost efficiency.

Arsenic contained in crude oil can cause problems to the processes and systems for producing olefins using steam cracking, particular in the product recovery section. The product recovery section can include, e.g., an acetylene converter containing an acetylene converter catalyst, a methylacetylene/propadiene converter (“MAPD”) containing an MAPD catalyst, a pygas first stage hydroprocessing reactor containing a di-olefins hydrogenation catalyst, and a pygas second stage hydroprocessing reactor containing a hydrodesulfurization catalyst. Arsenic, if present in such streams, especially at elevated quantity, can degrade or poison the respective catalysts. Moreover, the olefins products, particularly propylene, can be contaminated by the arsenic present in the crude feed if not properly managed during the production process.

There is a need for improved processes for producing olefins such as ethylene and propylene from an arsenic-containing hydrocarbon feed using steam cracking. This disclosure satisfies this and other needs.

### SUMMARY

In a first aspect, this disclosure provides a process for producing light olefins from a hydrocarbon feed comprising arsenic at an initial quantity. The process can comprise (I) introducing the hydrocarbon feed to a desalter to produce a desalted hydrocarbon feed having a first quantity of arsenic, where the first quantity is preferably from 60% to 95% of the initial quantity. The process can further comprise (II) heating the desalted hydrocarbon feed (preferably in a convection zone of a steam cracker) to form a preheated hydrocarbon feed. The process can further comprise (III) introducing the preheated hydrocarbon feed to a flash separation vessel to produce an overhead fraction having a second quantity of arsenic, and a bottoms fraction having a third quantity of arsenic, wherein the second quantity is preferably from 50% to 90% of the first quantity. The process can further comprise (IV) introducing the overhead fraction and steam to a radiant section of the steam cracker operated under steam cracking conditions to produce a steam cracked effluent having the second quantity of arsenic. The process can further comprise (V) separating the steam cracked effluent to obtain a steam cracker tar (“STC”), a steam cracker gas oil (“STGO”), a naphtha cut, and a process gas stream.

### BRIEF DESCRIPTION OF THE DRAWINGS

So that the manner in which the above recited features of the disclosure can be understood in detail, a more particular description of the disclosure, briefly summarized above, may be had by reference to implementations, some of which are illustrated in the appended drawings. It is to be noted, however, that the appended drawings illustrate only typical implementations of this disclosure and are therefore not to be considered limiting of scope, for the disclosure may admit to other equally effective implementations.

FIG. 1 schematically illustrates a first portion of a process/system 90 for producing light olefins by steam cracking including a desalter, a flash separation drum, a steam cracker, a tar knock-out drum, and a primary fractionator, according to one or more embodiments.

FIG. 2 schematically illustrates a second portion of the process/system 90 for separating a naphtha cut produced in FIG. 1.

FIG. 3 schematically illustrates a third portion of the process system 90 for recovering various products such as ethylene and propylene from a process gas stream produced in FIG. 1.

To facilitate understanding, identical reference numerals have been used, where possible, to designate identical elements that are common to the Drawings. It is contemplated that elements and features of one implementation may be beneficially incorporated in other implementations without further recitation.

### DETAILED DESCRIPTION

The present disclosure relates to methods, apparatuses, and systems for producing light olefins from an arsenic-containing hydrocarbon feed, preferably a heavy hydrocarbon-containing hydrocarbon feed such as crude, using steam cracking. As growth rates in demand for light olefins increase and steam crackers grow in size, it has become increasingly desirable to utilize heavy hydrocarbon feedstocks including various crude oil streams as a feed for steam cracker production of light olefins. Heavy hydrocarbon and crude oil feedstocks contain arsenic contaminants that may affect the steam cracking process, such as by deactivating catalysts or reducing the value of the product olefins. The management of such contaminants allows for more cost effective processing within the operating requirements of steam crackers and products that meet stringent specifications.

### Definitions

The term “Cn hydrocarbon,” where n is a positive integer, means any hydrocarbon comprising n carbon atom(s) per molecule, or any mixture thereof. The term “Cn+ hydrocarbon,” where n is a positive integer, means any hydrocarbon having at least n carbon atom(s) per molecule, and any mixture thereof. The term “Cn− hydrocarbon,” where n is a positive integer, means any hydrocarbon comprising no more than n number of carbon atom(s) per molecule, and any mixture thereof. “Cm-Cn hydrocarbon,” where m and n are positive integers and n>m, means any hydrocarbon comprising k carbon atoms per molecule, where k is a positive integer and m≤k≤n, and any mixture thereof.

The term “olefin” or “alkene” interchangeably means a hydrocarbon comprising at least one carbon to carbon double bond in its molecule. Non-limiting examples of olefins include ethylene, propylene, 1-butene, 2-butene, styrene, and diolefins. “Diolefin” means a hydrocarbon comprising two carbon to carbon double bonds. Non-limiting examples of diolefins include propadiene, 1,2-butadiene, 1,3-butadiene, 1,2-pentadiene, 1,3-pentadiene, 2,3-pentadiene, and 1,4-pentadiene.

The term “alkyne” means a hydrocarbon comprising at least one carbon to carbon triple bond in its molecule. Non-limiting examples of alkynes include acetylene (HC≡CH, ethyne), methylacetylene (HC≡C—CH₃, prop-1-yne), but-1-yne; but-2-yne, and but-1,3-diyne.

The term “steam cracker tar” or “SCT” means (a) a mixture of hydrocarbons having one or more aromatic components and optionally (b) non-aromatic and/or non-hydrocarbon molecules, the mixture being derived from hydrocarbon pyrolysis and having a Too of about 290° C. or greater. The SCT can include about 50 wt % or greater (e.g., 75 wt % or greater, such as 90 wt % or greater), based on the weight of the SCT, of hydrocarbon molecules (including mixtures and aggregates thereof) having (i) one or more aromatic components and (ii) a molecular weight of about C15 or greater. SCT generally has a metals content of about 103 ppmw or less, based on the weight of the SCT. The SCT metals content is generally far less than that found in crude oil (or crude oil components) of the same average viscosity.

The term “tar heavies” means a product of hydrocarbon pyrolysis, the TH having an atmospheric boiling point of about 565° C. or greater and includes about 5 wt % or more of molecules having a plurality of aromatic cores based on the weight of the product. The TH are typically solid at about 25° C. and generally include the fraction of SCT that is not soluble in a 5:1 (v/v) ratio of n-pentane: SCT at 25° C. TH generally includes asphaltenes and other high molecular weight molecules.

The term “non-volatile components” are the fraction of a hydrocarbon stream with a nominal boiling point about 590° C. or greater, as measured by ASTM D- 6352-98 or D-2887. Non-volatile components may be further limited to components with a boiling point of about 760° C. or greater. The boiling point distribution of a hydrocarbon stream may be measured by gas chromatograph distillation according to the methods described in ASTM D-6352-98 or D2887, extended by extrapolation for materials above 700° C. Non-volatile components may include coke precursors, which are moderately heavy and/or reactive molecules, such as multi-ring aromatic compounds, which can condense from the vapor phase and then from coke under the operating conditions encountered in the present process.

The term “T₅₀” means the temperature, determined according to the boiling point distribution described above, at which 50 weight percent of a particular hydrocarbon sample has reached its boiling point. Likewise, “T₉₀”, “T₉₅” and “T₉₈” mean the temperature at which 90, 95, or 98 weight percent of a particular sample has reached its boiling point. Nominal final boiling point means the temperature at which 99.5 weight percent of a particular sample bas reached its boiling point.

The term “steam cracker” is interchangeable with “thermal pyrolysis unit”, “pyrolysis furnace”, or just “furnace.” Steam, although optional, may be added for a variety of reasons, such as to reduce hydrocarbon partial pressure, to control residence time, and/or to decrease coke formation. In at least one embodiment, the steam may be superheated, such as in the convection section of the furnace, and/or the steam may be sour or treated process steam.

In this disclosure, “wppm,” “ppmw,” and “ppm by weight” interchangeably mean parts per million by mass. Thus, an arsenic concentration of x wppm or x ppmw in a given stream or material means a concentration of x parts per million by mass of arsenic atoms, based on the total mass of the stream or material in question. In this disclosure, “wppb,” “ppbw,” and “ppb by weight” interchangeably mean parts per billion by mass. Thus, an arsenic concentration of x wppb or x ppbw in a given stream or material means a concentration of x parts per billion by mass of arsenic atoms, based on the total mass of the stream or material in question.

The addition of steam at various points in the process is not detailed in every embodiment described. It is further noted that any of the steam added may include sour or treated process steam and that any of the steam added, whether sour or not, may be superheated. Superheating is common when the steam comes from sour water.

### The Steam Cracker Feed

The hydrocarbon feed may include relatively high molecular weight hydrocarbons (heavy hydrocarbon), such as those which produce a relatively large amount of steam cracker naphtha (SCN), steam cracker gas oil (SCGO), and steam cracker tar during steam cracking. The heavy hydrocarbon typically includes C5+ hydrocarbon, which may include one or more of SCGO and residues, gas oils, heating oil, jet fuel, fuel oil, diesel, kerosene, coker naphtha, SCN, catalytically cracked naphtha, hydrocrackate, reformate, raffinate reformate, Fischer-Tropsch liquids, Fischer-Tropsch gases, distillate, crude oil, atmospheric pipestill bottoms, vacuum pipestill streams including bottoms, gas oil condensates, heavy non-virgin hydrocarbon streams from refineries, vacuum gas oils, heavy gas oil, naphtha contaminated with crude, atmospheric residue, heavy residue, C4/residue admixture, naphtha residue admixture, gas oil residue admixture, low sulfur waxy residue, atmospheric residue, and heavy residue. It may be advantageous to use a heavy hydrocarbon feedstock including economically advantaged, minimally processed heavy hydrocarbon streams containing non-volatile components and coke precursors. The hydrocarbon feed can have a nominal final boiling point of about 315° C. or greater, such as about 400° C. or greater, about 450° C. or greater, or about 500° C. or greater.

The hydrocarbon feed may include one or more relatively low molecular weight hydrocarbon (light hydrocarbon). Light hydrocarbon typically includes substantially saturated hydrocarbon molecules having fewer than five carbon atoms, e.g., ethane, propane, and mixtures thereof. Although hydrocarbon feeds of light hydrocarbon typically produce a greater yield of C2 unsaturates (ethylene and acetylene) than do hydrocarbon feeds comprising heavy hydrocarbon, and the steam cracking of light hydrocarbon generally produces less SCN, SCGO, and steam cracker tar, the use of heavy hydrocarbon is of increasing interest due to lower costs and higher availability. The relative amounts of light hydrocarbon (typically in the vapor phase) and heavy hydrocarbon (typically in the liquid phase) in the hydrocarbon feed can be from 100 wt % light hydrocarbon to 100 wt % heavy hydrocarbon, although typically there is about 1 wt % or more heavy hydrocarbon present in a hydrocarbon feed. For example, the hydrocarbon feed can include about 1 wt % or more of heavy hydrocarbon, based on the weight of the hydrocarbon feed, such as about 25 wt % or more, about 50 wt % or more, about 75 wt % or more, about 90 wt % or more, or about 99 wt % or more.

The hydrocarbon feed also includes arsenic as a contaminant. Such arsenic may be present in the hydrocarbon feed in the form of elemental arsenic, inorganic arsenic compounds, organic arsenic compounds, and mixtures and combinations thereof. The arsenic contaminants can be or can include arsenic, arsine (AsH₃), one or more aliphatic arsines, one or more arsenites, one or more arsenates, one or more arsenides, salts thereof, or any combination or mixtures thereof. Non-limiting exemplary aliphatic arsines include RₓAsH₍₃₋ₓ₎, where x is 1, 2, or 3, and each R is independently an alkyl, an aryl (such as phenyl), or other organic groups. Non-limiting exemplary arsenites include (RO)₃As and non-limiting exemplary arsenates include (RO)₃AsO, where each R is independently an alkyl, an aryl (such as phenyl), or other organic groups. Thus, non-limiting exemplary organic arsenic compounds include ethylarsine (C₂H₅AsH₂), phenylarsine (C₆H₆AsH₂), ethyl arsenite ((C₂H₅O)₃As), ethyl arsenate ((C₂H₅O)₃AsO), and any combination thereof. Exemplary arsenides include metal arsenides, such as sodium arsenide, potassium arsenide, cobalt arsenide, iron arsenide, gallium arsenide, or any combination thereof.

For the purpose of this disclosure, the quantity of arsenic in a feed, a stream, a material, or a product is expressed as the quantity of the atoms of arsenic, regardless of the form in which the atoms of arsenic are present therein, unless specified otherwise or the context clearly indicates otherwise. The quantity of arsenic can be expressed in mass or in mole. Occasionally in this disclosure, a concentration or arsenic in a feed, a stream, a material, or a product may be described. For the purpose of this disclosure, any concentration of arsenic is expressed as the concentration of the atoms of arsenic in the feed, stream, material, or product in question, regardless of the form and oxidation state in which the atoms of arsenic are present therein, unless specified otherwise of the context clearly indicates otherwise.

The hydrocarbon feed comprises arsenic at a non-negligible initial quantity, which can be expressed in mass or in mole. The hydrocarbon feed can comprise arsenic at a concentration from, e.g., 0.01, 0.02, 0.04, 0.05, 0.06, 0.08 wppm, to 0.1, 0.2, 0.4, 0.5, 0.6, 0.8 wppm, to 1.0, 1.2, 1.4, 1.5, 1.6, 1.8, 2.0 wppm, based on the total mass of the hydrocarbon feed. In a preferred embodiment, the hydrocarbon feed can comprise or can be a crude. The initial quantity of arsenic in the hydrocarbon feed, in mass, can be calculated by multiplying the arsenic concentration and the total mass of the hydrocarbon feed.

### Desalter

Because of the desirably low concentration of arsenic and sodium in the radiant section of steam crackers, one or more desalters may be included to remove arsenic contaminants, salts, and particulate matter from the hydrocarbon feed prior to steam cracking. While acceptable arsenic contaminants, salt, and/or particulate matter concentrations vary with furnace design, the addition of a desalter may be desired when arsenic contaminants and/or sodium chloride is greater than a few ppm by weight of the hydrocarbon feed, and can further depend on the operating conditions of a particular feed. Desalting removes a portion of the arsenic contaminants, salts, and/or particulates to reduce catalyst poisoning, corrosion, fouling, and contamination issues.

In a typical desalting process, wash water (or fresh water, or deionized water) is mixed with a heated hydrocarbon feed to produce a water-in-oil emulsion, which in turn extracts salt, brine and particulates from the oil. The wash water used to treat the hydrocarbon feed may be derived from various sources and the water itself may be, for example, recycled refinery water, recirculated process water, clarified water, purified process water, sour water stripper bottoms, overhead condensate, boiler feed water, clarified river water or from other water sources or combinations of water sources. Salts in water are measured in parts per thousand by weight (ppt) and typically can include from fresh water (less than 0.5 ppt), brackish water (0.5-30 ppt), saline water (greater than 30 ppt to 50 ppt) to brine (greater than 50 ppt). Although deionized water may be used to favor exchange of salt from the crude into the aqueous solution, de-ionized water is not normally required to desalt crude oil feedstocks although it may be mixed with recirculated water from the desalter to achieve a specific ionic content in either the water before emulsification or to achieve a specific ionic strength in the final emulsified product. Wash water rates may be from about 5% and about 7% by volume of the total crude charge, but may be higher or lower dependent upon the crude oil source and quality. A variety of water sources may be combined as determined by cost requirements, supply, salt content of the water, salt content of the hydrocarbon feed, and other factors specific to the desalting conditions such as the size of the separator and the degree of desalting required.

In one or more embodiments, the hydrocarbon feed having an initial quantity of arsenic is introduced to or otherwise passed through the desalter to produce a desalted hydrocarbon feed having a first quantity of arsenic, which is less than the initial quantity. While it may be desirable to remove in the desalter as much as possible of the arsenic from the initial quantity to reduce negative impact of arsenic on the downstream processes and equipment, preferably the desalting conditions at the desalter are selected such that the first quantity is in a range from, e.g., 60%, 65%, 70%, 75%, to 80%, 90%, or 95% (more preferably 80% to 90%) of the initial quantity, to achieve a balance of cost and benefit. Where the first quantity is below 60% of the initial quantity, the desalter can be inordinately large, consumes overly large amount of wash water, thereby compromising the overall economy of the process. Where the first quantity is higher than 95% of the initial quantity, overly large amount of arsenic can be fed into the steam cracker, resulting in overly high arsenic load in various steams in the downstream processes, causing overly fast degrading and/or poisoning of the downstream catalyst(s) exposed such As-containing streams. The desalted hydrocarbon feed can have an arsenic concentration ranging from, e.g., 0.01, 0.02, 0.04, 0.05, 0.06, 0.08 wppm, to 0.1, 0.2, 0.4, 0.5, 0.6, 0.8, 0.9 wppm, based on the total mass of the desalted hydrocarbon feed.

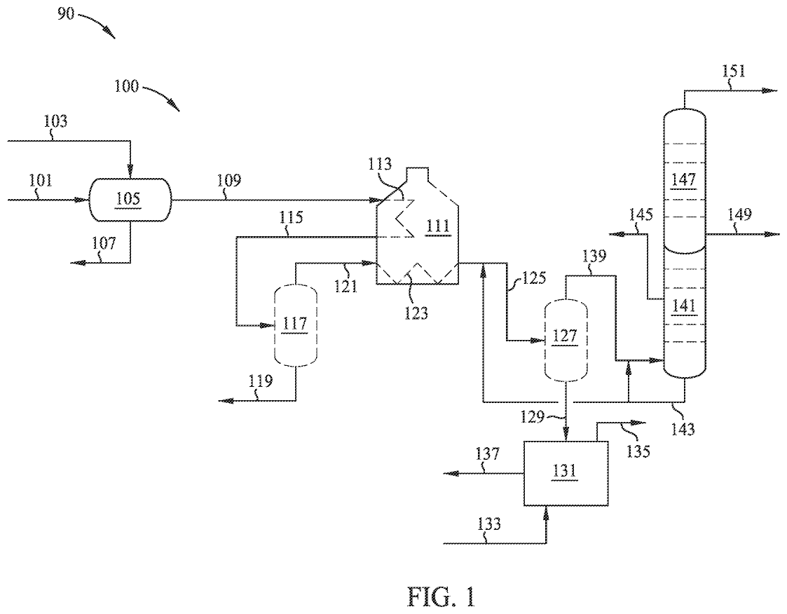

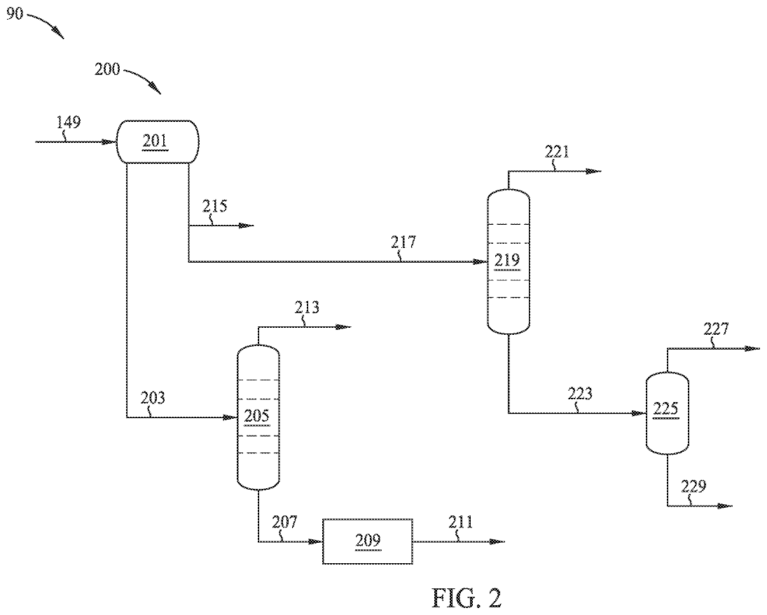

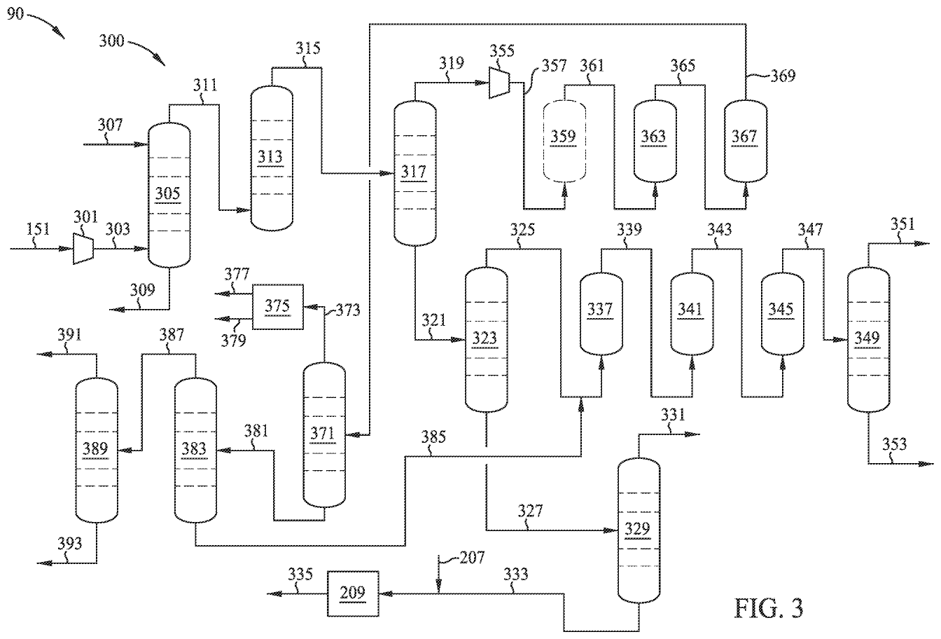

FIG. 1 depicts a partial schematic view of a process system 90 which is used to produce light olefins while reducing or eliminating arsenic contaminants from a hydrocarbon feed 101, according to one or more embodiments. The process system 90 contains a hydrocarbon steam cracking and fractionating system 100, as depicted in FIG. 1, a Pygas and water separation and purification system 200, depicted in FIG. 2, and a light hydrocarbon recovery system 300, depicted in FIG. 3.

In one or more embodiments, the process system 90 includes the hydrocarbon steam cracking and fractionating system 100, as depicted in FIG. 1. A salty emulsion is produced by combining the heavy hydrocarbon via the hydrocarbon feed 101 and wash water via line 103 within a desalter 105. The heavy hydrocarbon and water are mixed and then separated producing (1) process water that is transferred via line 107 and (2) desalted hydrocarbon feed that is removed from the desalter 105 via line 109. During the separation phase of the desalting process, an emulsion phase of varying composition and thickness may form at the interface of the oil and aqueous layers. If unresolved, these emulsions may carry-over with the desalted crude oil or carry-under into the aqueous layer. If carried-over, the emulsions may lead to fouling of downstream equipment and disruption of the downstream fractionation process. If carried-under, the emulsions may disrupt the downstream process water treatment process. Consequently, refiners typically desire to either control the formation/growth of these emulsions or remove the emulsions from desalter units and, using an additional processing step, resolve the emulsion into its constituent parts (e.g., oil, water and solids) to allow for reuse and/or disposal of the oil, water, and solids.

Methods for resolving emulsions may include gravitational or centrifugal methods. In a gravity method, the emulsion is allowed to stand in the separator and the density difference between the oil and the water causes the water to settle through and out of the oil by gravity. In the centrifugation method, the stable emulsion is moved from the de-salter unit to a centrifuge (not shown) which separates the emulsion into separate water, oil and solids. The gravity method requires the use of time-intensive, and thus inefficient, settling tanks as well as costly methods for disposing of the partially resolved emulsion, while the centrifugation method requires large centrifuges that are costly to build and operate.

Another method for resolving emulsions is the application of an electric field within the desalter. The application of an electric field may force water droplets to coalesce. Large electrocoalesced water droplets settle under gravity and penetrate through the oil/bulk-resolved-water interface to immerse into the resolved bulk water phase at the bottom of the desalter.

Certain hydrocarbon feed contaminants, including arsenic contaminants and/or natural surfactants (asphaltenes and resins) and finely divided solid particles (e.g., less than 5 microns), stabilize the emulsion phase and cause the emulsion to persist in the desalter unit (the emulsion layer in the desalter is commonly referred to as the rag layer). The persistent emulsion problem is prevalent in the processing of a hydrocarbon feed including crude oil because of high solids content. Hydrocarbon feeds with high solids contents are typically not preferred since the presence of the solids, often with particle sizes under 5 microns, may act to stabilize the emulsion and the oil/bulk-resolved-water interface, leading to a progressive increase in the depth of the rag layer. The persistent existence of a rag layer may be due to the inability of electrocoalesced droplets to break the oil/bulk-resolved-water interface. The rag layer in the desalter typically contains a high concentration of oil, residual water, suspended solids and salts which, in a typical example, might be about 70% v/v water, 30% v/v oil, with 5,000-8,000 pounds per thousand barrels (PTB) (about 14 g/L to about 23 g/L) solids, and 200-400 PTB (about 570 mg/L to about 1,100 mg/L) salts. The aqueous phase contains salts from the hydrocarbon feed.

One method of decreasing the size and effect of the persistent emulsified layer (rag layer) is the addition of demulsifiers. One suitable method for the addition of demulsifiers in the desalting process is described in U.S. Pub. No. 2016/0208176, incorporated by reference. Demulsifiers commonly used in the processing of hydrocarbon feeds that include heavy hydrocarbons may be useful in the desalting process although the desalting process may not be reliant on the specific demulsifier chosen. Demulsifiers may be one or more of: polyethyleneimines, polyamines, succinated polyamines, polyols, ethoxylated alcohol sulfates, long chain alcohol ethoxylates, long chain alkyl sulfate salts, e.g., sodium salts of lauryl sulfates, epoxies, di-epoxides (which may be ethoxylated and/or propoxylated). The addition of demulsifiers may be useful in the desalting of hydrocarbon feeds containing high levels of particulates or asphaltenes, which tend to stabilize the rag layer.

The desalted oil phase forms a top layer, which is continuously removed as desalted hydrocarbon feed via line 109 and the resolved bulk water accumulates in the bottom of the desalter and is continuously removed as process water via line 107 (FIG. 1). The process water may be sent for deionization and recycling or used with or without further processing in other refinery processes.

### Steam Cracker

Steam cracking is carried out in at least one steam cracker (furnace), the steam cracker includes a radiant section and a convection section (discussed in further detail below). The steam cracker may have a flash separation vessel integrated by fluid connection between the convection section and the radiant section. The radiant section can include fired heaters, and flue gas from combustion carried out with the fired heaters travels upward from the radiant section through the convection section and then away as flue gas. As shown in FIG. 1, desalted hydrocarbon feed via line 109 first enters a steam cracker 111 in the convection section (upper portion) and through line 113 where it is preheated by exposure to the flue gases in the convection section to produce a preheated hydrocarbon feed. Preheated hydrocarbon feed via line 115 enters a flash separation vessel 117 (also referred to as a separation pot or a K-Pot), which heats and separates liquid and vapor portions of the preheated hydrocarbon feed into a residual product stream that is transferred via line 119. Separation vessel also forms steam cracking feed (overhead fraction) that is transferred via line 121 to the steam cracker 111 and through one or more radiant section tubes 123 in the radiant section (lower portion) of the steam cracker 111 for pyrolysis (cracking) to produce steam cracked effluent that is transferred to line 125 for further processing.

### Steam Cracker Convection Section

The desalted hydrocarbon feed (via line 109) is first preheated, preferably (but not required) in the convection section line 113 within the convection section of the steam cracker 111. The heating of the desalted hydrocarbon feed may include indirect contact (within the convection line) of the feed in the convection section of the steam cracker with hot flue gases from the radiant section of the furnace. The preheating of the desalted hydrocarbon feed can be accomplished, for example, by passing the desalted hydrocarbon feed through bank of heat exchange tubes located within the convection section of the steam cracker. The preheated hydrocarbon feed may have a temperature from about 150 ° C. to about 260° C., such as about 160° C. to about 230° C., or about 170° C. to about 220° C.

The preheated desalted hydrocarbon feed may be combined with additional amount £ of steam and the resultant mixture may be subjected to additional preheating in the convection section. In certain aspects, the weight ratio of the additional amount of steam to desalted hydrocarbon feed can be from, e.g., about 0.1 to about 1, such as about 0.2 to about 0.6.

### Flash Separation Vessel

The stream cracker 111 may have integrated therewith one or more flash separation vessels 117, which is a vapor/liquid separation device (sometimes referred to as flash pot or flash drum). Such flash separation vessels are suitable when the preheated hydrocarbon feed includes about 0.1 wt % or more of asphaltenes based on the weight of the hydrocarbon components of the convection product stream, e.g., about 5 wt % or more. Upgrading the preheated hydrocarbon feed through vapor/liquid separation may be accomplished through flash separation vessels or other suitable means. Examples of suitable flash separation vessels include those disclosed in U.S. Pat. Nos. 6,632,351; 7,090,765; 7,097,758; 7,138,047; 7,220,887; 7,235,705; 7,244,871; 7,247,765; 7,297,833; 7,311,746; 7,312,371; 7,351,872; 7,488,459; and 7,578,929; and 7,820,035, which are incorporated by reference herein.

Where a flash separation vessel is integrated with the steam cracker, at least a portion of the preheated hydrocarbon feed is in the vapor phase. The preheated hydrocarbon feed (via line 115) is transferred to and flashed in one or more flash separation vessel 117, in order to separate the liquid phase and the vapor phase and at least a portion of the high molecular-weight molecules, such as asphaltenes, remaining in the liquid phase. A bottoms fraction, from the liquid phase, can be conducted away from the flash separation vessel 117 as a residual product stream via line 119. The bottoms fraction may include, for example, greater than about 10 wt % of the asphaltenes in the preheated hydrocarbon feed. The overhead fraction, from the vapor phase, can be conducted to the steam cracker 111 as a steam cracking feed via line 121. In at least one embodiment, the overhead fraction (steam cracking feed) from the flash separation vessel can be subjected to further heating in the convection section near convection line 113, and is then introduced as steam cracking feed via crossover piping (not shown) into the radiant section (e.g., the radiant line 123).

One advantages of having a flash separation vessel downstream of the convection section and upstream of the radiant section is an increased breadth of hydrocarbon types available to be used directly, without pretreatment, as the hydrocarbon feed 101. For example, the addition of a flash separation vessel downstream of convection line 113 allows for hydrocarbon feed 101 to contain about 50% or greater, such as about 75% or greater, or about 90% or greater, up to 95%, 98%, or even 100% of crude oils or heavy hydrocarbons.

The use of a flash separation vessel upstream of the radiant section of the steam cracker may allow operation with undesired contaminants in the hydrocarbon feed or desalted hydrocarbon feed because contaminants in the vapor phase may be kept within desired limits. The flash separation vessel may remove most or all of the remaining salt and particulate matter in the liquid phase, for example when less than about 98% of the hydrocarbon is a vapor at the inlet of the flash separation vessel.

The flash separation vessel may operate at a temperature from about 315° C. to about 510° C. and/or a pressure from about 275 kPa to about 1,400 kPa, such as, a temperature from about 430° C. to about 480° C., and/or a pressure from about 700 kPa to about 760 kPa.

Typically, a vapor phase overhead fraction is separated from the hydrocarbon feed in the flash separation vessel forming a steam cracking feed. The steam cracking feed is conducted away from the flash separation vessel to the radiant line in the radiant section of the steam cracker for pyrolysis. The liquid-phase separated from the hydrocarbon feed as the bottoms fraction can be conducted away from the flash separation vessel, e.g., for storage and/or further processing. The operation conditions in the flash separation vessel can be chosen such that the overhead fraction comprises a second quantity of arsenic, and the bottoms fraction comprises a third quantity of arsenic. The sum total of the second quantity and the third quantity of arsenic is equivalent to the second quantity of arsenic in the desalted hydrocarbon feed, if the desalted hydrocarbon feed is the only hydrocarbon feed fed into the flash separation vessel. While it may be desirable as much as possible of the arsenic in the first quantity makes its way into the bottoms fraction, i.e., to form the third quantity of arsenic, preferably, however, the conditions in the flash separation vessel are chosen such that the second quantity, i.e., the quantity of arsenic in the overhead fraction, ranges from, e.g., 50%, 55%, 60%, 65%, 70%, to 75%, 80%, 85%, 90% (preferably 70% to 90%), of the first quantity of arsenic present in the desalted hydrocarbon feed, to achieve a balance of cost and benefit.

In one or more embodiments, the preheated hydrocarbon feed via line 115 is introduced into the flash separation vessel 117 to form the steam cracking feed via line 121 having an arsenic concentration preferably no greater than 500 wppm, based on the total mass of the steam cracking feed. Thus, preferably, the steam cracking feed in line 121 supplied to the steam cracker radiant section can have arsenic at a concentration from, e.g., 50, 80, 100, 150, 200, 250, to 300, 350, 400, 450, 500, wppm, based on the total mass of the steam cracking feed.

### Steam Cracker Radiant Section

The steam cracking feed is transferred to the radiant section, where the steam cracking feed is indirectly exposed (in the radiant line) to the combustion carried out by the burners. As shown in FIG. 1, steam cracking feed via line 121 is introduced into radiant line 123, where at least a portion of the hydrocarbon in the steam cracking feed is pyrolysed to produce steam cracked effluent, including C2+ olefins, which is transferred to line 125. The steam cracking feed is typically in the vapor phase at the inlet of the radiant coils, e.g., about 90 wt % or greater of the steam cracker feed is in the vapor phase, such as about 95 wt % or greater, or about 99 wt % or greater.

Steam cracking conditions (pyrolysis conditions) may include exposing the steam cracking feed in the radiant line 123 to a temperature (measured at the outlet of the radiant line) of about 400° C. or greater, such as, from about 400° C. to about 1,100° C., and a pressure of about 10 kPa or greater, and a steam cracking residence time from about 0.01 second to 5 seconds. For example, the steam cracking conditions can include one or more of (i) a temperature of about 760° C. or greater, such as from about 760° C. to about 1,100° C., or from about 790° C. to about 880° C., or for hydrocarbon feeds containing light hydrocarbon from about 760° C. to about 950° C.; (ii) a pressure of about 50 kPa or greater, such from about 60 kPa to about 500 kPa, or from about 90 kPa to about 240 kPa; and/or (iii) a residence time from about 0.1 seconds to about 2 seconds. The steam cracking conditions may be sufficient to convert at least a portion of the steam cracking feed's hydrocarbon molecules to C2+ olefins by pyrolysis.

As a result of the pyrolysis reactions of hydrocarbons in the radiant section, the steam cracked effluent generally includes molecular hydrogen, CH₄, C2 hydrocarbons such as ethane, ethylene, and acetylene, C3 hydrocarbons such as propylene, propane, methylacetylene, propadiene, C4 hydrocarbons including alkanes, mono olefins, diolefins, and alkynes; C5-C10 hydrocarbons, and C11+ hydrocarbons. The high-temperature steam cracked effluent exiting the radiant section of the steam cracker is typically immediately quenched, and separated to produce a steam cracker tar, a steam cracker gas oil, a steam cracker naphtha cut, and a process gas stream rich in C4-hydrocarbons and molecular hydrogen. The process gas stream can be separated to produce, among others, an ethylene product stream, and a propylene product stream. Contaminants present in the steam cracking feed may undergo various chemical reactions in the radiant section, producing various contaminant molecules such as mercaptans, CO₂, H₂S, COS, and the like.

Arsenic-containing contaminants in the steam cracking feed may undergo chemical reactions in the radian section, producing a series of arsenic-containing compounds in the steam cracked effluent, and upon separation, distributed at various quantities in the steam cracker tar, the steam cracker gas oil, the steam cracker naphtha cut, and the process gas stream. For example, arsine (AsH₃) may be produced in the radiant section, present in the steam cracked effluent, and upon separation of the quenched steam cracked effluent, be distributed predominantly into the process gas stream. The steam cracking conditions in the radiant section and separation process conditions can be selected, based at least partly on the second quantity of arsenic in the steam cracking feed and overall composition of the steam cracking feed and desired products, such that: (i) that the steam cracker tar and the steam cracker gas oil, in total, comprise a fourth quantity of arsenic; (ii) the process gas stream comprises a fifth quantity of arsenic; and (iii) the naphtha cut comprises a sixth quantity of arsenic. Preferably, the fourth quantity is from 60% to 85% (e.g., 60%, 62%, 64%, 65%, 66%, 68%, 70%, 72%, 74%, 75%, 76%, 78%, 80%, 82%, 84%, or 85%) of the second quantity. Preferably, the fifth quantity is from 1% to 10% (e.g., 1%, 2%, 3%, 4%, 5%, 6%, 7%, 8%, 9%, 10%) of the second quantity. Preferably, the sixth quantity is from 10% to 30% (e.g., 10%, 12%, 14%, 15%, 16%, 18%, 20%, 22%, 24%, 25%, 26%, 28%, or 30%) of the second quantity. We have found that the fourth quantity, the fifth quantity, and the sixth quantity can be controlled by controlling the steam cracking conditions (temperature, pressure, steam/hydrocarbon ratio, and the residence time in the radiant section, e.g.) in the steam cracker at a given second quantity. The processes of this disclosure are highly advantageous in that, by controlling the steam cracking conditions in the light of the second quantity, a significant portion of the arsenic present in the desalted hydrocarbon feed is converted into components in the steam cracker that are, upon separation, distributed into the steam cracker tar and steam cracker gas oil, where the presence of arsenic at the combined fourth quantity would not pose an issue for downstream processing and/or use thereof. In addition, by controlling the steam cracking conditions in the light of the second quantity, only a very small fraction of the arsenic present in the desalted hydrocarbon feed is converted into components in the steam cracker that are, upon separation, distributed into the process gas stream, where the presence of arsenic at the fifth quantity can be predicted and practically and conveniently managed as described below. Moreover, by controlling the steam cracking conditions in the light of the second quantity, only a minor portion of the arsenic present in the desalted hydrocarbon feed is converted into components in the steam cracker that are, upon separation, distributed into the naphtha cut, where the presence of arsenic at the combined sixth quantity can be predicted and practically and conveniently managed as described below.

### Tar Knock Out Drum

The steam cracking process produces molecules which tend to combine to form high molecular weight materials known as steam cracked tar. The steam cracked effluent is a combination of desirable C1-C10 species, steam cracked gas oil (C10-C17), and steam cracked tar. Steam cracked tar is a high-boiling point, viscous, reactive material that can foul equipment under certain conditions. In general, feedstocks containing higher boiling materials tend to produce greater quantities of tar. The formation of steam cracked tar, after the steam cracked effluent leaves the radiant line in the steam cracker, can be decreased by rapidly reducing the temperature of the effluent to a level at which the tar-forming reactions are greatly slowed. The rapid reduction in temperature of the steam cracked effluent may be achieved in one or more steps and using one or more methods and is referred to as quenching. The steam cracked effluent can be quenched by various methods such as contacting with cooled hydrocarbon (direct quench), or, alternatively, the steam cracked effluent can be rapidly cooled in heat exchangers.

As shown in FIG. 1, a tar knock-out drum 127, accepts steam cracked effluent (via line 125) and separates the effluent into steam cracked tar (which is transferred to line 129) and tar light products stream (which is transferred to line 139). The steam cracked effluent may undergo cooling or quenching before being introduced to the tar knock-out drum or as it is introduced to the tar knock-out drum.

In at least one embodiment, the steam cracked effluent is quenched by rapid cooling through one or more heat exchangers (not shown). Generally, the effluent leaving the first heat exchanger may remain at a temperature above the hydrocarbon dew point (the temperature at which the first drop of liquid condenses) of the steam cracked effluent. For a typical hydrocarbon feed containing heavy hydrocarbons under cracking conditions, the hydrocarbon dew point of the steam cracked effluent may be from about 375° C. to about 650° C., such as from about 480° C. to about 600° C. Above the hydrocarbon dew point, the fouling tendency is relatively low, because vapor phase fouling is generally not severe, and there is little to no liquid present that could cause fouling. The steam cracked effluent may be further cooled by an additional heat exchanger, direct quench before reaching the tar knock-out drum, or direct quench within the tar knock-out drum.

In at least one embodiment, the steam cracked effluent is subjected to direct quench at a point between the radiant line 123 and the tar knock-out drum 127. The quench is accomplished by contacting the steam cracked effluent with a liquid quench stream, in lieu of, or in addition to the treatment with transfer line exchangers. Where employed in conjunction with at least one transfer line exchanger, the quench liquid may be introduced at a point downstream of the transfer line exchanger(s). Suitable quench liquids include liquid quench oil, such as those obtained by a downstream tar knock-out drum, clean fuels unit, or primary fractionator, pyrolysis fuel oil and water, which can be obtained from various suitable sources, e.g., the condensed dilution steam.

After passage through the direct quench and/or transfer line heat exchanger(s), the quenched steam cracked effluent is fed to at least one tar knock-out drum (a separation vessel). where the steam cracked tar is separated from the tar light product stream. The temperature of the quenched steam cracked effluent entering the tar knock-out drum should be at a sufficiently low temperature that the tar separates. Tar separates rapidly at temperatures of about 350° C. or less, such as from about 200° C. to about 350° C. or from about 240° C. to about 320° C.

The tar knockout drum 127 can be a simple vessel, lacking distillation plates or stages. If desired, multiple knock-out drums may be connected in parallel such that individual drums can be taken out of service and cleaned while the plant is operating. The steam cracked tar removed in the tar knock-out drum typically has an initial boiling point ranging from about 150° C. to about 320° C., typically, about 200° C. or greater.

In at least one embodiment, a purge stream is introduced to the tar knock-out drum 127 to reduce liquid-vapor contact. Typically, the purge stream is selected from steam, inert gas such as nitrogen, and substantially non-condensable hydrocarbons, such as those obtained from steam cracking, examples of which include cracked gas and tail gas.

Quenching of the steam cracked effluent may also be accomplished within the knock-out drum. Quenching within the tar knock-out drum may be accomplished by feeding the steam cracked effluent through a cool (less than 350° C.) quench fluid (such as one or more of the quench fluids described above). A cool quench fluid may be created by feeding a stream of steam cracked tar taken from the bottom of the tar knock-out drum through a suitable heat exchanger (e.g., a shell-and-tube exchanger, spiral wound exchanger, airfin, or double-pipe exchanger) and recycling the cooled steam cracked tar stream to the tar knock-out drum. In at least one embodiment, sufficient cooled steam cracked tar is recycled to reduce the temperature of the tar recycle from about 280° C. to about 150° C. The rate of asphaltene and tar formation in the line 125 and the tar knock-out drum 127 is greatly reduced at temperatures about 280° C. or less as compared to the higher temperatures of the steam cracker effluent when leaving the radiant line. In another embodiment, the recycling suffices to reduce viscosity of the tar removed from the tar knock-out drum to an extent sufficient to meet viscosity specifications, in the absence or reduction of an added externally sourced light blend stock otherwise necessary in the absence of said recycling. In another embodiment, the cooled tar is introduced to the separation vessel so as to provide an average temperature for tar within the separation vessel of about 175° C. or less, such as about 150° C. or less. Quenching methods may be adjusted to prevent the formation of asphaltenes. It may be possible to prevent formation of up to about 70 wt % of asphaltenes through quenching the steam cracked effluent via line 125 in the tar knock-out drum 127.

In one or more embodiments, the steam cracked effluent via line 125 is introduced into the tar knock-out drum 127, and the steam cracked tar via line 129 is separated from the tar light product stream via line 139.

### Clean Fuels Unit

The steam cracked tar from the tar knock-out drum can be further processed in a clean fuels unit. The clean fuels unit may be a hydroprocessing unit in which steam cracked tar, a utility fluid (optional), treat gas including hydrogen, and catalyst are combined under hydroprocessing conditions to produce clean fuels product (upgraded steam cracked tar) having improved blending characteristics with other heavy hydrocarbons such as fuel oil. The clean fuels unit may further remove sulfur and other impurities to provide a clean fuels product that is compatible with fuel oils. As shown in FIG. 1, a clean fuels unit 131 accepts a steam cracked tar 129 from a tar knock-out drum 127 and a lean amine stream via line 133. After hydroprocessing and removal of sulfurous and other impurities, the clean fuels unit 131 produces a rich amine stream via line 135 and a clean fuels product stream via line 137.

Steam cracked tar can be a highly aromatic product with a Tso boiling point similar to a vacuum gas oil and/or a vacuum resid fraction. Steam cracked tar can be difficult to process using a fixed bed reactor because various molecules within the steam cracked tar are highly reactive, leading to fouling and operability issues. Such processing difficulties can be further complicated, for example, by the high viscosity of the feed, the presence of coke fines, and/or other properties related to the composition of steam cracked tar.

The use of a utility fluid in hydroprocessing steam cracked tar may lessen deposit formation. The use of a utility fluid may provide a clean fuels product with a decreased viscosity, a decreased atmospheric Tso or Too boiling point, and increased hydrogen content over that of the SCT, resulting in improved compatibility with fuel oil blend-stocks. Additionally, hydroprocessing the SCT in the presence of utility fluid may produce fewer undesirable byproducts and the rate of increase in reactor pressure drop is lessened, which may increase run-length during hydroprocessing of SCT. The utility fluid may be a portion of the clean fuels product that is recycled. Suitable processes for SCT hydroprocessing with a utility fluid and recycling a portion of the product stream as a utility fluid are disclosed in U.S. Pat. Nos. 9,777,227 and 9,809,756 and in International Patent Application Publication No. WO 2013/033590, which are incorporated herein by reference.

The relative amounts of utility fluid and SCT during hydroprocessing are generally from about 20 wt % to about 95 wt % of the SCT and from about 5 wt % to about 80 wt % of the utility fluid, based on total weight of utility fluid plus SCT. For example, the relative amounts of utility fluid and SCT during hydroprocessing can be (i) from about 20 wt % to about 90 wt % of the SCT and from about 10 wt % to about 80 wt % of the utility fluid, or (ii) from about 40 wt % to about 90 wt % of the SCT and from about 10 wt % to about 60 wt % of the utility fluid. In an embodiment, the utility fluid: SCT weight ratio can be about 0.01 or greater, e.g., from about 0.05 to about 4, such as from about 0.1 to about 3, or from about 0.3 to about 1.1.

A utility fluid may include a solvent having significant aromatics content and generally, the utility fluid may also include a mixture of multi-ring compounds. The rings can be aromatic or non-aromatic and can contain a variety of substituents and/or heteroatoms. For example, the utility fluid can contain about 40 wt % or greater, about 45 wt % or greater, about 50 wt % or greater, about 55 wt % or greater, or about 60 wt % or greater, based on the total weight of the utility fluid, of aromatic and non-aromatic ring compounds. The utility fluid can have an ASTM D86 10% distillation point of about 60° C. or greater and a 90% distillation point of about 350° C. or less. Optionally, the utility fluid (which can be a solvent or mixture of solvents) has an ASTM D86 10% distillation point of about 120° C. or greater, 140° C. or greater, or about 150° C. or greater and/or an ASTM D86 90% distillation point of about 300° C. or less.

The hydroprocessing is carried out in the presence of hydrogen by (i) combining molecular hydrogen with the tar stream and/or utility fluid upstream of the hydroprocessing and/or (ii) conducting molecular hydrogen to the hydroprocessing stage in one or more conduits or lines. Although relatively pure molecular hydrogen can be utilized for the hydroprocessing, it is generally desirable to utilize a “treat gas” which contains sufficient molecular hydrogen for the hydroprocessing and optionally other species (e.g., nitrogen and light hydrocarbons such as methane) which generally do not adversely interfere with or affect either the reactions or the products. The treat gas may contain about 50 vol % or greater of molecular hydrogen, such as about 75 vol % or greater, based on the total volume of treat gas conducted to the hydroprocessing stage.

The amount of molecular hydrogen supplied to the hydroprocessing stage can be from about 300 SCF/B (standard cubic feet per barrel) (53 S m³/m³) to about 5,000 SCF/B (890 S m³/m³), in which B refers to barrel of feed to the hydroprocessing stage (e.g., tar stream plus utility fluid). For example, the amount of molecular hydrogen can be from about 1,000 SCF/B (about 178 S m³/m³) to about 3,000 SCF/B (about 534 S m³/m³). The amount of molecular hydrogen required to hydroprocess the SCT is less if the SCT contains higher amounts of C6+ olefin, for example, vinyl aromatics. Optionally, higher amounts of molecular hydrogen may be supplied, for example, when the tar stream contains relatively higher amounts of sulfur.

Hydroprocessing catalysts can be utilized for hydroprocessing the SCT in the presence of the utility fluid, such as those specified for use in resid and/or heavy oil hydroprocessing. Examples of suitable hydroprocessing catalysts include one or more of KF860 available from Albemarle Catalysts Company LP, Houston TX; Nebula® Catalyst, such as Nebula® 20, available from the same source; Centera® catalyst, available from Criterion Catalysts and Technologies, Houston TX, such as one or more of DC-2618, DN-2630, DC-2635, and DN-3636; Ascent® Catalyst, available from the same source, such as one or more of DC-2532, DC-2534, and DN-3531; and FCC pre-treat catalyst, such as DN3651 and/or DN3551, available from the same source.

Other suitable hydroprocessing catalysts include those comprising (i) one or more bulk metals and/or (ii) one or more metals on a support. The metals can be in elemental form or in the form of a compound. In one or more embodiments, the hydroprocessing catalyst includes one or more metals from any of Groups 5 to 10 and/or 15 of the Periodic Table of the Elements (tabulated as the Periodic Chart of the Elements, The Merck Index, Merck & Co., Inc., 1996). Examples of such catalytic metals include vanadium, chromium, molybdenum, tungsten, manganese, technetium, rhenium, iron, cobalt, nickel, ruthenium, palladium, rhodium, osmium, iridium, platinum, alloys thereof, or any combination thereof. In one or more examples, each of the catalysts can be or include a nickel sulfide catalyst, a nickel molybdenum catalyst, a cobalt molybdenum catalyst, or any combination thereof.

The metal may be incorporated into or deposited on a support including porous materials. The support can include one or more refractory oxides, porous carbon-based materials, zeolites, or combinations thereof suitable refractory oxides include, e.g., alumina, silica, silica-alumina, titanium oxide, zirconium oxide, magnesium oxide, and mixtures thereof. Suitable porous carbon-based materials include activated carbon and/or porous graphite. Examples of zeolites include, e.g., Y-zeolites, beta zeolites, mordenite zeolites, ZSM-5 zeolites, and ferrierite zeolites. The support can be heat-treated at temperatures from about 400° C. to about 1,200°C, such as from about 450° C. to about 1,000° C., or from about 600° C. to about 900° C., prior to impregnation, incorporation, or deposition with the metals. In at least one embodiment, the catalyst is heat treated after combining the support with one or more of the metals, heat treating of the catalyst and support together may take place at a temperature from about 150° C. to about 750° C., such as from about 200° C. to about 740° C., or from about 400° C. to about 730° C. The heat treatment may take place in the presence of hot air and/or oxygen-rich air at a temperature of about 400° C. or greater in order to remove volatile matter, and convert at least a portion of the metals to their corresponding metal oxide.

The hydroprocessing is generally accomplished under conditions for carrying out one or more of hydrocracking (including selective hydrocracking), hydrogenation, hydrotreating, hydrodesulfurization, hydrodenitrogenation, hydrodemetallation, hydrodearomatization, hydroisomerization, or hydrodewaxing of the SCT. Hydroprocessing of the SCT in the presence of the utility fluid, treat gas, and catalyst can occur in one or more hydroprocessing stages, the stages comprising one or more hydroprocessing vessels or zones downstream of the steam cracker and optionally downstream of the tar knock-out drum.

The SCT generally contacts the hydroprocessing catalyst in the vessel or zone, in the presence of the utility fluid and molecular hydrogen. Catalytic hydroprocessing conditions can include, e.g., exposing the combined utility fluid and SCT to a temperature from about 50° C. to about 500° C., such as from about 200° C. to about 450° C., from about 220° C. to about 430° C., from about 300° C. to about 500° C., from about 350° C. to about 430° C., or from about 350° C. to about 420° C. proximate to the molecular hydrogen and hydroprocessing catalyst. Liquid hourly space velocity (LHSV) of the combined utility fluid and SCT may be from about 0.1 h⁻¹ to about 30 h⁻¹, or about 0.4 h⁻¹ to about 25 h⁻¹, or about 0.5 h⁻¹ to about 20 h⁻¹. In some embodiments, LHSV is about 5 h⁻¹ or greater, or about 10 h⁻¹ or greater, or about 15 h⁻¹ or greater. Molecular hydrogen partial pressure during the hydroprocessing can be from about 0.1 MPa to about 8 MPa, or about 1 MPa to about 7 MPa, or about 2 MPa to about 6 MPa, or about 3 MPa to about 5 MPa. In some embodiments, the partial pressure of molecular hydrogen is about 7 MPa or less, about 6 MPa or less, about 5 MPa or less, about 4 MPa or less, about 3 MPa or less, about 2.5 MPa or less, or about 2 MPa or less. The hydroprocessing conditions can include, a pressure from about 1.5 mPa to about 13.5 mPa, or from about 2 mPa to about 12 mPa, or from about 2 mPa to about 10 mPa. The hydroprocessing conditions may further include a molecular hydrogen consumption rate of about 53 standard cubic meters/cubic meter (S m³/m³) to about 445 S m³/m³ (300 SCF/B to 2500 SCF/B, where the denominator represents barrels of the tar stream, e.g., barrels of SCT).

When hydroprocessed SCT or clean fuels product has improved properties compared to those of SCT, the clean fuels product 137 can be suitable for use as a fuel oil blending component. For example, the clean fuels product generally exhibits improved viscosity, solubility number, and insolubility number over the SCT and a lower sulfur content than SCT. Blending of the clean fuels product with other heavy hydrocarbons can be accomplished with little or no asphaltene precipitation, even without further processing of the clean fuels product prior to the blending.

The clean fuels product may be separated into overhead, mid-cut, and bottoms by a separation device, e.g., one or more of distillation towers, vapor-liquid separators, splitters, fractionation towers, membranes, or absorbents. Describing the separated portions of the clean fuels product as overhead, mid-cut, and bottoms is not intended to preclude separation methods other than fractionating in a distillation tower. The overhead may include from about 0 wt % to about 20 wt % of the clean fuels product. The mid-cut may include from about 20 wt % to about 70 wt % of the clean fuels product. The bottoms may include from about 20 wt % to about 70 wt % of the clean fuels product.

In certain aspects, at least a portion of the overhead includes unused treat gas and may be recycled after removing undesirable impurities including H₂S and NH₃. A vapor portion of the overhead may be directed through one or more amine towers which receive lean amine and conduct away rich amine. The upgraded vapor product may be recycled as a portion of the treat gas. Furthermore molecular hydrogen may be added to recycled portion to maintain the level of hydrogen entering the clean fuels unit as necessary for hydroprocessing the steam cracked tar.

### Primary Fractionator

As mentioned above, the tar light product stream is separated in a primary fractionator. Returning to FIG. 1, tar light product stream, via line 139, includes liquid-phase and gas-phase products and is conducted to a separation stage, e.g., a primary fractionator 141 and a quench tower 147, for separation into a plurality of hydrocarbon product streams. The hydrocarbon product streams may include one or more of a (i) heavy hydrocarbon recycle stream, which is transferred to line 143, including about 90 wt % or greater of SCT, based on the weight of the bottoms (heavy hydrocarbon recycle stream), the SCT having a boiling point of about 290° C. or greater and contain molecules and mixtures thereof having a molecular weight of about 212 g/mole or greater, (ii) steam cracked gas oil (“SCGO”), which is sent away via line 145, the SCGO including about 90 wt % or greater of C10-C17 species based on the weight of the SCGO and having an T90 boiling point from about 200° C. to about 290° C., (iii) a naphtha cut (also known as pyrolysis gasoline (Pygas)) stream, which is transferred via line 149 and contains C5-C10 hydrocarbons, also referred to as steam cracked naphtha, and (iv) a process gas stream, which is transferred via line 151.

Suitable primary fractionators and associated equipment are described in U.S. Pat. No. 8,083,931 and U.S. Pub. No. 2016/0376511, which are incorporated by reference herein. Additional stages for removing heat (such as one or more transfer line heat exchangers) and removing tar (such as tar drums) can be located in or upstream of the primary fractionator, if desired.

The tar light product stream via line 139 is introduced to the primary fractionator 141 in a way that decreases contact with the vapor in the fractionator. If the tar light product stream were simply sprayed into the vapor space, the tar light product stream would tend to heat up as a result of mixing with the large quantity of hot vapor present and would also absorb light components from the vapor, which might not be desired. Instead, the tar light product stream can be introduced near or preferably just below the liquid-vapor interface in the bottom of the primary fractionator. Introducing the tar light product stream below the vapor liquid interface ensures the stream is cooled to the desired temperature and decreases the absorption of light components. An optional baffle placed above the vapor-liquid interface can reduce contact of the tar light product stream with hot vapor.

The liquid portion of the primary fractionator is composed of heavy hydrocarbons and tar which can be released from the bottom of the primary fractionator as a heavy hydrocarbon recycle stream via line 143. The viscosity of the heavy hydrocarbon recycle stream taken from the bottom of the primary fractionator can be controlled by the addition of a light blend stock, which may be added directly to the bottom of the primary fractionator and provide cooling of the heavy hydrocarbon recycle stream. Alternately, the light blend may be added downstream of the primary fractionator as part of the heavy hydrocarbon recycle stream. Such light blends may include steam cracked gas oil, distillate quench oil and cat cycle oil and are characterized by viscosity at a temperature of 93° C. of about 1,000 centistokes (cSt) or less, such as about 500 cSt or less, or about 100 cSt or less. The heavy hydrocarbon recycle stream may be recycled and combined with the steam cracked effluent before the steam cracked effluent enters the tar knock-out drum (e.g., recycling the heavy hydrocarbon recycle stream to line 125). The heavy hydrocarbon recycle stream can also be recycled to combine with the tar light product stream. In either manner, the heavy hydrocarbon recycle stream can provide liquid cooling in the separations that occur in the tar knock-out drum or the primary fractionator.

The steam cracked gas oil may be condensed out of the vapor phase within the primary fractionator 141. The remaining vapor constitutes a vapor phase effluent.

The vapor phase effluent can be passed through the overhead of the primary fractionator into a quench tower (such as the quench tower 147), where the vapor is rapidly cooled (quenched) as the vapor passes through water (vapor or liquid). The water can be obtained from a variety of sources, for example, recycled refinery water, recirculated process water, clarified fresh water, purified process water, sour water stripper bottoms, overhead condensate, boiler feed water, or from other water sources or combinations of water sources. Water is commonly recycled from downstream oil water separators, sour water separators, and Pygas strippers. The quench tower condenses at least a portion of Pygas present in the vapor-phase effluent. Condensed Pygas and heated quench water are withdrawn from a location proximate to the bottom of the quench tower as a naphtha cut.

The process gas stream (quench tower gaseous overhead) is collected from the overhead of the quench tower, such as from the overhead of the quench tower 147 via line 151. When utilizing the specified steam cracking feed and the specified steam cracker conditions, the process stream can include, for example, about 10 wt % or greater of C2+ olefin, about 1 wt % or greater of C6+ aromatic hydrocarbon, about 0.1 wt % or greater of diolefin, saturated hydrocarbon, molecular hydrogen, acetylene, CO₂, aldehyde, and C1+ mercaptan. The process gas stream may be directed to a recovery train for recovering C2 to C4 olefins, among other things.

In one or more embodiments, the tar light product stream via line 139 is introduced into the primary fractionator 141 to produce at least the naphtha cut via line 149 and the process gas stream via line 151.

### Light Hydrocarbon Recovery Train

The process gas stream has a fifth quantity of arsenic, which can be preferably from 1% to 10%, more preferably from 1% to 5%, such as 1%, 2%, 3%, 4%, or 5% of the second quantity described above.

In one or more embodiments, the process system 90 includes the light hydrocarbon recovery system 300, as depicted in FIG. 3. The process gas stream via line 151 (from FIG. 1) from the overhead of the quench tower may be compressed in one or more stages of gas compressors 301. A significant amount of the arsenic in the process gas stream may be in the form of arsine (AsH₃). The compressed process gas are transferred via line 303 to an amine tower 305 where the compressed light hydrocarbons are purified. The amine tower may accept a light amine stream 307 including aqueous solutions of one or more of ethanolamine, diethanolamine, methyldiethanolamine, diisopropanolamine, diglycolamine, and other amines. The amine tower removes acid gases, e.g., hydrogen sulfide and carbon dioxide, within rich amine stream (in line 309). For further removal of acid gases, the amine-treated process gas stream after exiting the amine tower may be passed through line 311 to a caustic tower 313, which may include aqueous hydroxide solutions, e.g., aqueous sodium hydroxide. The caustic tower removes remaining acid gases including hydrogen sulfide and carbon dioxide and also some weak acid gases (e.g., mercaptans). The removal of acid gases generates a low sulfur process gas stream 315.

The low sulfur hydrocarbon stream, comprising molecular hydrogen, methane, ethane, ethylene, acetylene, propane, propylene, methylacetylene, propadiene, C4 hydrocarbons and C5 hydrocarbons, can be separated by using distillation columns operated at low temperatures to recover, among others, a tail gas stream rich in CH₄, an ethylene product stream, a propylene product stream, and C4 product streams. Desirably the propylene product stream comprises arsenic at a concentration no greater than 0.05 wppb. Many different configurations of the distillation columns may be used to recover the products. The recovering process can include a step of passing a C3-hydrocarbon-containing stream comprising C3 alkynes/dienes through an arsine removal bed to produce a treated C3-hydrocarbon-containing stream having an arsine concentration no greater than 2 wppb, based on the total weight of the treated C3-hydrocarbon-containing stream, followed by passing the treated C3-hydrocarbon-containing stream through a first hydrogenation reactor to contact an alkyne/diene hydrogenation catalyst in the presence of molecular hydrogen to convert at least a portion of the C3 alkyne/dienes to propylene. In certain embodiments, the first hydrogenation reactor is a front-end acetylene converter including an acetylene conversion catalyst therein, in which case the C3-hydrocarbon-containing stream can comprise H₂, CH₄, ethane, ethylene, acetylene, propane, propylene, methylacetylene, and propadiene. In a front-end acetylene converter, acetylene is selectively hydrogenated to form ethylene as well. In certain other embodiments, the first hydrogenation reactor is a methylacetylene/propadiene (“MAPD”) converter including a MAPD conversion catalyst therein, in which case the C3-hydrocarbon-containing stream can consist essentially of propane, propylene, methylacetylene, and propadiene, in addition to added H₂. Arsenic present in the treated C3-hydrocarbon-containing stream can accumulate in the alkyne/diene hydrogenation catalyst in the first hydrogenation reactor, leading to degrading and poisoning of the catalyst overtime. The arsine removal bed is used to protect the alkyne and/or diene hydrogenation catalyst. In certain embodiments of the processes of this disclosure, the process includes a step of determining a run-length of the arsine removal bed at a given arsenic removal capacity or an arsenic removal capacity of the arsine removal bed at a given run-length, based on at least one of the second quantity and the fifth quantity. As described above, by controlling the steam cracking conditions in the steam cracker, the fifth quantity can be controlled and predicted at a given second quantity. One therefore can calculate a run-length of the arsine removal bed at a given arsenic removal capacity or an arsenic removal capacity of the arsine removal bed at a given run-length, based on at least one of the second quantity and the fifth quantity. The ability to predict the capacity and/or run-length of the arsenic removal bed in order to protect the alkyne and/or diene hydrogenation catalyst allows one to optimize the design and operation of the arsine removal bed and the first hydrogenation reactor, which represents another significant advantage of such embodiments of this disclosure.

In certain non-limiting embodiments, the low sulfur hydrocarbon stream via line 315 may be separated in a fractionator 317 into C1-C2 hydrocarbons (with some C3+) removed through line 319 and C3+ hydrocarbons removed through line 321. For example, the process gas stream via line 151 can be purified to the low sulfur hydrocarbon stream via line 315 which is introduced to the (second) fractionator 317 to produce the C1-C2 hydrocarbon stream via line 319 and the C3+ hydrocarbon stream via line 321. The C3+ hydrocarbons are passed through line 321 to a fractionator 323 which separates C3 products into line 325 and C4+ products into line 327. The C4+ products in line 327 are again fractionated in a fractionator 329 into C4 product stream via line 331 and C5+ hydrocarbons via line 333. The C5+ fractions in line 207 (from the primary fractionator) and from line 333 are combined and may be passed through the gasoline hydrogenation unit 209 to produce various gasoline products that are transferred via line 335.

The C3 products from the fractionator 323 are transferred through line 325 to be purified in columns that may include (i) a methanol/COS bed 337, then through line 339 to (ii) an arsine bed 341, and through line 343 to (iii) an MAPD converter 345 for hydrogenation. For example, the C3+ hydrocarbon stream 321 is eventually passed through the arsine bed 341 to produce the reduced-arsine C3+ hydrocarbon stream via line 343. Thereafter, the reduced-arsine C3+ hydrocarbon stream via line 343 through the MAPD converter 345 to produce the purified C3 stream via line 347.

The reduced-arsine C3+ hydrocarbon stream via line 343 and/or the purified C3 stream via line 347 can have an arsenic concentration of less than 2 wppb, such as about 0.01 wppb, about 0.1 wppb, or about 0.5 wppb to about 0.8 wppb, about 1 wppb, about 1.2 wppb, about 1.5 wppb, about 1.7 wppb, or about 1.8 wppb. For example, the reduced-arsine C3+ hydrocarbon stream via line 343 and/or the purified C3 stream via line 347 have an arsenic concentration of about 0.01 wppb to about 1.8 wppb, about 0.01 wppb to about 1.5 wppb, about 0.01 wppb to about 1 wppb, about 0.01 wppb to about 0.5 wppb, about 0.1 wppb to about 1.8 wppb, about 0.1 wppb to about 1.5 wppb, about 0.1 wppb to about 1 wppb, about 0.1 wppb to about 0.5 wppb, about 0.5 wppb to about 1.8 wppb, about 0.5 wppb to about 1.5 wppb, or about 0.5 wppb to about 1 wppb.

Subsequently, the purified C3 hydrocarbons pass through line 347 to enter a C3 splitter 349 (e.g., a fractionator) that separates propylene (transferred via line 351) from propane (transferred via line 353). The propane of line 353 may be recycled for further cracking or used in other refinery processes. The propylene stream via line 351 has an arsenic concentration of less than 0.05 wppb.

The C1-C2 portions from the fractionator 317 are transferred through line 319 to a compressor 355 and further compressed (this is downstream of compressor 301). From the compressor 355, the C1-C2 products pass through line 357 to a series of purification systems which may include (i) a sulfur-compound removal bed 359 (e.g., a mercaptan and carbonyl sulfide removal bed), then through line 361 to (ii) an arsine bed 363, and then through line 365 to (iii) a C2 acetylene converter 367. For example, the C1-C2 hydrocarbon stream via line 319 is eventually passed through the arsine bed 363 from line 361 to produce a reduced-arsine C1-C2 hydrocarbon stream via line 365. Thereafter, the reduced-arsine C1-C2 hydrocarbon stream via line 365 is passed through the acetylene converter 367 to produce the purified C1-C2 stream via line 369.

The reduced-arsine C1-C2 hydrocarbon stream via line 365 and/or the purified C1-C2 stream via line 369 have an arsenic concentration of less than 2 wppb, such as about 0.01 wppb, about 0.1 wppb, or about 0.5 wppb to about 0.8 wppb, about 1 wppb, about 1.2 wppb, about 1.5 wppb, about 1.7 wppb, or about 1.8 wppb. For example, the reduced-arsine C1-C2 hydrocarbon stream via line 365 and/or the purified C1-C2 stream via line 369 have an arsenic concentration of about 0.01 wppb to about 1.8 wppb, about 0.01 wppb to about 1.5 wppb, about 0.01 wppb to about 1 wppb, about 0.01 wppb to about 0.5 wppb, about 0.1 wppb to about 1.8 wppb, about 0.1 wppb to about 1.5 wppb, about 0.1 wppb to about 1 wppb, about 0.1 wppb to about 0.5 wppb, about 0.5 wppb to about 1,8 wppb, about 0.5 wppb to about 1.5 wppb, or about 0.5 wppb to about 1 wppb.

Thereafter, the purified C1-C2 stream is passed through line 369 to a demethanizer 371 for further separation. The overhead of the demethanizer 371 includes methane which is passed through line 373 to a cold box 375 in order to separate methane via line 377 from residual hydrogen via line 379. The methane of line 377 may be used as fuel gas and/or steam cracked again for the production of syngas and hydrogen. Hydrogen of line 379 can be recycled to the clean fuels unit as a hydrogen source in one or more hydroprocessing units. The C2 stream (the bottoms portion of the demethanizer) may be passed through line 381 into a fractionator 383 which removes residual C3+ and recycles the C3+ hydrocarbons through line 385 to line 325 which provides effluent to a methanol/COS bed 337. The overhead fraction of the fractionator 383 includes C2 hydrocarbons which are passed through line 387 to a C2 splitter 389 to separate ethylene (transferred via line 391) from ethane (transferred via line 393). Ethane may be recycled for further cracking or used in other refinery processes. The ethylene stream via line 391 can have a negligible concentration of arsenic.

Each of the arsine beds 341, 363 independently contains one or more materials for removing arsine and/or other arsenic compounds, materials, or contaminants. For example, each of the arsine beds 341, 363 independently contains lead oxide which is used to remove arsine and/or other arsenic contaminants from the process stream upstream of converters containing catalyst beds, such as the MAPD converter 345 and/or the acetylene converter 367.

### Naphtha Cut Processing

The naphtha cut has a sixth quantity of arsenic, which can be preferably from 20% to 30%, such as 20%, 22%, 24%, 25%, 26%, 28%, or 30%, of the second quantity. In certain embodiments, the processes of this disclosure include: (VIII) recovering a C5+ hydrocarbon stream comprising C5+ diolefins from the naphtha cut; and (IX) contacting the C5+ hydrocarbon stream with molecular hydrogen in the presence of a C5+ diolefins hydrogenation catalyst in a second hydrogenation reactor to convert at least a portion of the C5+ diolefins into C5+ olefins and to produce a C5+ diolefins-abated C5+ hydrocarbon stream comprising C5+ olefins. Preferably, the conditions in the second hydrogenation reactor are selected such that the C5+ diolefins-abated C5+ hydrocarbon stream comprises a seventh quantity of arsenic, and the seventh quantity is 50% to 70% (e.g., 50%, 55%, 60%, 65%, or 70%) of the sixth quantity. In preferred embodiments, the C5+ diolefins hydrogenation catalyst comprises nickel sulphide which has desirable arsenic tolerance capable of removing a significant amount of arsenic during its run-length from the C5+ hydrocarbon stream.

In certain preferred embodiments, the processes of this disclosure further comprise: (X) determining a run-length of the second hydrogenation reactor at a given arsenic removal capacity or an arsenic removal capacity of the second hydrogenation reactor at a given run-length based on at least one of the second quantity, the sixth quantity, and the seventh quantity. As described above, by controlling the steam cracking conditions in the steam cracker, the sixth quantity can be controlled and predicted at a given second quantity. One therefore can calculate a run-length of the second hydrogenation reactor at a given arsenic removal capacity of the C5+ diolefins hydrogenation catalyst or an arsenic removal capacity of the C5+ diolefins hydrogenation catalyst at a given run-length, based on at least one of the second quantity and the sixth quantity. The ability to predict the capacity and/or run-length of the C5+ diolefins hydrogenation catalyst allows one to optimize the design and operation of the second hydrogenation reactor, which represents another significant advantage of such embodiments of this disclosure.

In certain preferred embodiments, the processes of this disclosure further comprise: (XI) contacting the C5+ diolefins-abated C5+ hydrocarbon stream with molecular hydrogen in the presence of a hydrodesulfurization catalyst in a third hydrogenation reactor to convert at least a portion of the C5+ olefins into C5+ paraffins and to produce a hydrodesulfurized C5+ hydrocarbon stream. In certain preferred embodiments, the hydrodesulfurization catalyst comprises (i) nickel and molybdenum or (ii) cobalt and molybdenum, which has desirable arsenic tolerance capable of removing a significant amount of arsenic during its run-length from C5+ diolefins-abated C5+ hydrocarbon stream. In certain preferred embodiments, the conditions in the third hydrogenation reactor are selected such that the hydrodesulfurized C5+ hydrocarbon stream comprises an eighth quantity of arsenic, and the eighth quantity is 0.1% to 10% (e.g., 0.1%, 0.5%, 1%, 5%, or 10%) of the sixth quantity. In certain preferred embodiments, the processes of this disclosure further comprises (XII) determining a run-length of the third hydrogenation reactor at a given arsenic removal capacity or an arsenic removal capacity of the third hydrogenation reactor at a given run-length based on at least one of the second quantity, the sixth quantity, the seventh quantity, and the eighth quantity. As described above, by controlling the steam cracking conditions in the steam cracker, the sixth quantity can be controlled and predicted at a given second quantity. One therefore can calculate a run-length of the third hydrogenation reactor at a given arsenic removal capacity of the hydrodesulfurization catalyst or an arsenic removal capacity of the hydrodesulfurization catalyst at a given run-length, based on at least one of the second quantity the seventh quantity, and the eighth quantity. The ability to predict the capacity and/or run-length of the hydrodesulfurization catalyst allows one to optimize the design and operation of the third hydrogenation reactor, which represents another significant advantage of such embodiments of this disclosure.

In one or more embodiments, the process system 90 can include the Pygas and water separation and purification system 200, as depicted in FIG. 2. The naphtha cut via line 149 (also line 149 in FIG. 1) may be separated from water downstream in an oil and water separator 201 to form separated water and separated Pygas. The separated Pygas with some remaining water may be transferred via line 203 to a Pygas stripper 205 and further processed in the Pygas stripper 205. The Pygas may be taken from the bottoms portion of the Pygas stripper 205 and may include C5-C10 hydrocarbons and may be transferred via line 207 to a gasoline hydrogenation unit 209 to produce various gasoline products via line 211. Water and light hydrocarbons can be removed from the top of the Pygas stripper 205 and recycled via line 213 to the primary fractionator. Water that is removed in the Pygas stripper 205 may also be transferred to downstream processes or to process water treatment.

In one or more embodiment, the gasoline hydrogenation unit 209 can include one, two, three, or more stages for hydrogenating the diolefins to produce olefins. Arsenic contaminants can spend, poison, or otherwise reduce the reactivity of catalyst contained in the various stages of the gasoline hydrogenation unit 209. A portion of the arsenic contaminants have been removed from the hydrocarbon effluent (e.g., the Pygas via line 207) relative to the hydrocarbon effluent upstream from the Pygas and water separation and purification system 200, such as the hydrocarbon feed 101, or other hydrocarbon effluents in the hydrocarbon steam cracking and fractionating system 100.

In some embodiments, the gasoline hydrogenation unit 209 contains a first stage Pygas hydrotreater reactor with a first catalyst and a second stage Pygas hydrotreater reactor with a second catalyst. By using predetermined catalysts as the first and/or second stage catalysts, a tolerable level of catalyst poisoning by the arsenic contaminant (in the feed to the reactors) can provide acceptable run-lengths of the hydrogenation process. In one or more examples, the first catalyst is or includes a nickel sulfide catalyst in the first stage Pygas hydrotreater reactor and the second catalyst is or includes a nickel molybdenum catalyst, a cobalt molybdenum catalyst, or any combination thereof in the second stage Pygas hydrotreater reactor.

The separated water component from the oil water separator or the Pygas stripper may be recycled via line 215 to the desalter, quench tower, or used as steam in other refining processes. Alternatively, the separated water may be sent via line 217 to a sour water stripper 219 to remove hydrogen sulfide, ammonia, and other impurities. Sour water stripping generally provides for degasification of sour water removing light hydrocarbons and remaining hydrogen. The sour water stripper may be a steam-reboiled distillation column allowing for the overhead stripping of hydrogen sulfide and ammonia via line 221. Once the acid gas and ammonia have been removed, the clean water can be recycled (not shown) or transferred via line 223 to a dilution steam generator 225 to provide steam via line 227 to the steam cracker. The dilution steam generator may also produce process water that can be removed via line 229.

Overall, it has been found that removal of arsenic contaminants from hydrocarbon feeds including heavy hydrocarbons (which can be useful for steam cracking) can be accomplished by one or more of: (i) desalting the hydrocarbon feed, (ii) preheating the desalted hydrocarbon feed in the convection section of the steam cracker, (iii) vapor liquid separation in a flash separation vessel, (iv) pyrolysis in the radiant section of a steam cracker, (v) removal of tar heavies and steam cracked tar in a tar knock-out drum, and (v) fractionation of product feeds to separate a light hydrocarbon product stream. Some arsenic contaminants (e.g., arsine) may be carried through this process and can be removed in a downstream light hydrocarbon recovery train, such as with arsine beds. The combination of desalter, flash separation vessel, and tar knock-out drum with a steam cracker removes or reduces the majority of arsenic contaminants that cause catalyst poisoning and contamination of products.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of this disclosure, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and/or testing procedures to the extent they are not inconsistent with this text. As is apparent from the foregoing general description and the specific embodiments, while forms of this disclosure have been illustrated and described, various modifications can be made without departing from the spirit and scope of this disclosure. Accordingly, it is not intended that this disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below.

While this disclosure has been described with respect to a number of embodiments and examples, those skilled in the art, having benefit of this disclosure, will appreciate that other embodiments can be devised which do not depart from the scope and spirit of this disclosure.

What is claimed is:

1. A process for producing light olefins from a hydrocarbon feed comprising arsenic at an initial quantity, the process comprising:
(I) introducing the hydrocarbon feed to a desalter to produce a desalted hydrocarbon feed having a first quantity of arsenic, where the first quantity is from 60% to 95% of the initial quantity;
(II) heating the desalted hydrocarbon feed to form a preheated hydrocarbon feed;
(III) introducing the preheated hydrocarbon feed to a flash separation vessel to produce an overhead fraction having a second quantity of arsenic, and a bottoms fraction having a third quantity of arsenic, wherein the second quantity is from 50% to 90% of the first quantity;
(IV) introducing the overhead fraction and steam to a radiant section of the steam cracker operated under steam cracking conditions to produce a steam cracked effluent having the second quantity of arsenic; and
(V) separating the steam cracked effluent to obtain a steam cracker tar, a steam cracker gas oil, a naphtha cut, and a process gas stream.

2. The process of claim 1, wherein the steam cracked effluent comprises arsenic at a concentration of no greater than about 500 wppb, based on the total weight of the steam cracked effluent.

3. The process of claim 1, wherein step (V) comprises:
(Va) separating the steam cracked effluent in a tar knock-out drum to obtain the steam cracker tar and a drum upper effluent; and
(Vb) separating the drum upper effluent to obtain the steam cracker gas oil, the naphtha cut, and the process gas stream.

4. The process of claim 1, wherein:
steps (IV) and (V) are configured such that the steam cracker tar and the steam cracker gas oil, in total, comprise a fourth quantity of arsenic, the fourth quantity is from 70% to 80% of the second quantity.

5. The process of claim 1, wherein the process gas stream comprises a fifth quantity of arsenic, and the fifth quantity is from 1% to 5% of the second quantity.

6. The process of an claim 1, further comprising:
(VI) recovering an ethylene product stream and a propylene product stream from the process gas stream, wherein the propylene product stream comprises arsenic at a concentration no greater than 0.05 wppb, based on the total weight of the propylene product stream.

7. The process of claim 6, wherein step (VI) comprises:
(VIa) passing a C3-hydrocarbon-containing stream comprising C3 alkyne/dienes through an arsine removal bed to produce a treated C3-hydrocarbon-containing stream having an arsine concentration no greater than 2 wppb, based on the total weight of the treated C3-hydrocarbon-containing stream.

8. The process of claim 7, further comprising:
(VIb) passing the treated C3-hydrocarbon-containing stream through a first hydrogenation reactor to contact an alkyne/diene hydrogenation catalyst in the presence of molecular hydrogen to convert at least a portion of the C3 alkyne/dienes to propylene.

9. The process of claim 8, wherein the first hydrogenation reactor is a front-end acetylene converter or an MAPD convertor.

10. The process of claim 7, further comprising:
(VII) determining a run-length of the arsine removal bed at a given arsenic removal capacity or an arsenic removal capacity of the arsine removal bed at a given run-length based on at least one of the second quantity and the fifth quantity.

11. The process of claim 1, wherein the naphtha cut comprises a sixth quantity of arsenic, and the sixth quantity is from 20% to 30% of the second quantity.

12. The process of claim 1, further comprising:
(VIII) recovering a C5+ hydrocarbon stream comprising C5+ diolefins from the naphtha cut; and
(IX) contacting the C5+ hydrocarbon stream with molecular hydrogen in the presence of a C5+ diolefins hydrogenation catalyst in a second hydrogenation reactor to convert at least a portion of the C5+ diolefins into C5+ olefins and to produce a C5+ diolefins-abated C5+ hydrocarbon stream comprising C5+ olefins.

13. The process of claim 12, wherein conditions in the second hydrogenation reactor are selected such that the C5+ diolefins-abated C5+ hydrocarbon stream comprises a seventh quantity of arsenic, and the seventh quantity is 50% to 70% of the sixth quantity.

14. The process of claim 12, wherein the C5+ diolefins hydrogenation catalyst comprises nickel sulfide.

15. The process of claim 12, further comprising:
(X) determining a run-length of the second hydrogenation reactor at a given arsenic removal capacity or an arsenic removal capacity of the second hydrogenation reactor at a given run-length based on at least one of the second quantity, the sixth quantity, and the seventh quantity.

16. The process of claim 12, further comprising:
(XI) contacting the C5+ diolefins-abated C5+ hydrocarbon stream with molecular hydrogen in the presence of a hydrodesulfurization catalyst in a third hydrogenation reactor to convert at least a portion of the C5+ olefins into C5+ paraffins and to produce a hydrodesulfurized C5+ hydrocarbon stream.

17. The process of claim 16, wherein the hydrodesulfurization catalyst comprises (i) nickel and molybdenum or (ii) cobalt and molybdenum.

18. The process of claim 16, wherein conditions in the third hydrogenation reactor are selected such that the hydrodesulfurized C5+ hydrocarbon stream comprises an eighth quantity of arsenic, and the eighth quantity is 0.1% to 10% of the sixth quantity.

19. The process of claim 16, further comprising:
(XII) determining a run-length of the third hydrogenation reactor at a given arsenic removal capacity or an arsenic removal capacity of the hydrodesulfurization catalyst at a given run-length based on at least one of the second quantity, the sixth quantity, the seventh quantity, and the eighth quantity.

20. A process for producing light olefins from a hydrocarbon feed comprising arsenic at an initial quantity, the process comprising:
(I) introducing the hydrocarbon feed to a desalter to produce a desalted hydrocarbon feed having a first quantity of arsenic, where the first quantity is from 60% to 95% of the initial quantity;
(II) heating the desalted hydrocarbon feed to form a preheated hydrocarbon feed;
(III) introducing the preheated hydrocarbon feed to a flash separation vessel to produce an overhead fraction having a second quantity of arsenic, and a bottoms fraction having a third quantity of arsenic, wherein the second quantity is from 50% to 90% of the first quantity;
(IV) introducing the overhead fraction and steam to a radiant section of the steam cracker operated under steam cracking conditions to produce a steam cracked effluent having the second quantity of arsenic, wherein the steam cracked effluent comprises arsenic at a concentration of no greater than about 500 wppb, based on the total weight of the steam cracked effluent;
(V) separating the steam cracked effluent to obtain a steam cracker tar, a steam cracker gas oil, a naphtha cut, and a process gas stream; wherein steps (IV) and (V) are configured such that the steam cracker tar and the steam cracker gas oil, in total, comprise a fourth quantity of arsenic, the fourth quantity is from 70% to 80% of the second quantity; and
(VI) recovering an ethylene product stream and a propylene product stream from the process gas stream, wherein the propylene product stream comprises arsenic at a concentration no greater than 0.05 wppb, based on the total weight of the propylene product stream.

21. The process of claim 20, wherein the process gas stream comprises a fifth quantity of arsenic, and the fifth quantity is from 1% to 5% of the second quantity.

22. The process of claim 20, wherein step (VI) comprises:
(VIa) passing a C3-hydrocarbon-containing stream comprising C3 alkyne/dienes through an arsine removal bed to produce a treated C3-hydrocarbon-containing stream having an arsine concentration no greater than 2 wppb, based on the total weight of the treated C3-hydrocarbon-containing stream;
(VIb) passing the treated C3-hydrocarbon-containing stream through a first hydrogenation reactor to contact an alkyne/diene hydrogenation catalyst in the presence of molecular hydrogen to convert at least a portion of the C3 alkyne/dienes to propylene.

23. The process of claim 20, further comprising:
(VII) determining a run-length of the arsine removal bed at a given arsenic removal capacity or an arsenic removal capacity of the arsine removal bed at a given run-length, based on at least one of the second quantity and the fifth quantity.

24. The process of claim 20, wherein the naphtha cut comprises a sixth quantity of arsenic, and the sixth quantity is from 20% to 30% of the second quantity.

25. The process of claim 20, further comprising:
(VIII) recovering a C5+ hydrocarbon stream comprising C5+ diolefins from the naphtha cut; and
(IX) contacting the C5+ hydrocarbon stream with molecular hydrogen in the presence of a C5+ diolefins hydrogenation catalyst in a second hydrogenation reactor to convert at least a portion of the C5+ diolefins into C5+ olefins and to produce a C5+ diolefins-abated C5+ hydrocarbon stream comprising C5+ olefins;
wherein the C5+ diolefins hydrogenation catalyst comprises nickel sulfide, and conditions in the second hydrogenation reactor are selected such that the C5+ diolefins-abated C5+ hydrocarbon stream comprises a seventh quantity of arsenic, and the seventh quantity is 50% to 70% of the sixth quantity; and
(X) determining a run-length of the second hydrogenation reactor at a given arsenic removal capacity or an arsenic removal capacity of the second hydrogenation reactor at a given run-length, based on at least one of the second quantity, the sixth quantity, and the seventh quantity.

26. The process of claim 25, further comprising:
(XI) contacting the C5+ olefins in the C5+ diolefins-abated C5+ hydrocarbon stream with molecular hydrogen in the presence of a hydrodesulfurization catalyst in a third hydrogenation reactor to convert at least a portion of the C5+ olefins into C5+ paraffins and to produce a hydrodesulfurized C5+ hydrocarbon stream,
wherein conditions in the third hydrogenation reactor are selected such that the hydrodesulfurized C5+ hydrocarbon stream comprises an eighth quantity of arsenic, and the eighth quantity is 0.1% to 10% of the sixth quantity.

27. The process of claim 26, further comprising:
(XII) determining a run-length of the third hydrogenation reactor at a given arsenic removal capacity or an arsenic removal capacity of the third hydrogenation reactor at a given run-length based on at least one of the second quantity, the sixth quantity, the seventh quantity, and the eighth quantity.
